(19) United States
(12) Patent Application Publication
(10) Pub. No.: US 2022/0165397 A1
(43) Pub. Date: May 26, 2022

(54) PAPERLESS ONBOARDING METHOD AND SYSTEM

(71) Applicant: Blue Note Therapeutics, Inc., San Francisco, CA (US)

(72) Inventors: Laura Brown CHAVAREE, San Francisco, CA (US); Mark Wesley ELFERS, Simi Valley, CA (US); Geoffrey Spencer EICH, Camarillo, CA (US); Richard Adam LIT, Malibu, CA (US); Michael John MALECKI, Westlake Village, CA (US); Michael Antone MCKINLEY, Washington, UT (US)

(21) Appl. No.: 17/100,463

(22) Filed: Nov. 20, 2020

(51) Int. Cl. G16H 40/20; G16H 10/60; G06Q 10/10; G16H 20/10; H04L 29/06; G06F 21/62

(52) U.S. Cl. CPC G16H 40/20; G16H 10/60; G06Q 10/10; G06F 21/6245; H04L 63/083; H04L 63/102; G16H 20/10

(57) ABSTRACT

Examples described herein generally relate to a system and methods for registering a user with an online service. An application server receives, from a health care provider, first patient identifying information and second patient identifying information for a new account. The server transmits a message to the patient based on the second patient identifying information, the message including a unique access code for the patient. The server receives, from a client application via a secure link, a user access code and user identifying information. The server verifies that the user access code matches the unique access code and the user identifying information matches the first patient identifying information. The server requests, in response to the verifying, a new username and password via the client application. The server associates the new username and password with the new account, the online service, the first and second patient identifying information.

### TECHNICAL FIELD

The present disclosure relates to onboarding for an online service, and particularly to onboarding for digital therapeutics.

### BACKGROUND

Online services may be provided via an application. Typically, online services may collect a small amount of information about a user to facilitate communications or payment for the online service. Such information is generally not sensitive, not complicated, and not protected information. Further, usage of the application generally does not require permission from a third party. The user may download the application and sign up for service via the application by providing the necessary information.

Onboarding a new user for an online service that uses sensitive information about the user may present difficulties. For example, a digital medical therapeutic may be provided as an online service based on a prescription from a health care provider. The digital medical therapeutic may utilize sensitive information about the user such as medical or mental health conditions, psychological profiles, and Personally identifiable information (PII) associated with an electronic prescription. Such information may be protected by various privacy laws. In particular, the Health Insurance Portability and Accountability Act (HIPAA) requires certain protections for protected health information (PHI). Accordingly, onboarding for an online service such as a digital medical therapeutic may involve multiple parties and may require protection of information, which cannot be provided by conventional onboarding for online services.

Thus, there is a need in the art for improvements in onboarding for online services associated with a prescription or medical order.

### SUMMARY

The following presents a simplified summary of one or more implementations of the present disclosure in order to provide a basic understanding of such implementations. This summary is not an extensive overview of all contemplated implementations, and is intended to neither identify key or critical elements of all implementations nor delineate the scope of any or all implementations. Its sole purpose is to present some concepts of one or more implementations of the present disclosure in a simplified form as a prelude to the more detailed description that is presented later.

In an aspect, the disclosure provides a method of registering a user with an online service. The method may include receiving, from a health care provider, first patient identifying information for a new account and second patient identifying information for the new account. The method may include transmitting a message to the patient based on the second patient identifying information, the message including a unique access code for the patient. The method may include receiving, from a client application via a secure link, a user access code and user identifying information. The method may include verifying that the user access code matches the unique access code and the user identifying information matches the first patient identifying information. The method may include requesting, in response to the verifying, a new username and password via the client application. The method may include associating the new username and password with the new account, the online service, the first patient identifying information, and the second patient identifying information.

In an aspect, the first patient identifying information includes one or more of: a name, a sex, or a date of birth.

In an aspect, the second patient identifying information includes one or more of: a mailing address, a phone number, or an email address.

In an aspect, the method further includes receiving, via the health care provider, an indication of consent from the patient, wherein transmitting the message is in response to the consent from the patient. For example, the indication of consent may be one of a signed digital form, a digital copy of a signed form, or a recording of a telephone consent.

In an aspect, the unique access code is eight digits with no more than two repeating numbers.

In an aspect, the method further includes retiring the unique access code in response to confirming acceptance of the new username and password.

In an aspect, the enrollment request is an electronic prescription or physician order and the online service is a prescription-only digital therapeutic.

In an aspect, receiving the patient information includes: receiving from a health care provider an account creation request for a patient, the request including the first patient identifying information for a new account; and receiving, from a health care provider, an enrollment request for the online service, the enrollment request including the second patient identifying information for the new account.

In an aspect, the disclosure provides an apparatus for providing an online service to registered users. The apparatus may include a processor and a memory storing computer-executable instructions. The processor may be configured to receive, from a health care provider, first patient identifying information for a new account and second patient identifying information for the new account. The processor may be configured to transmit a message to the patient based on the second patient identifying information, the message including a unique access code for the patient. The processor may be configured to receive, from a client application via a secure link, a user access code and user identifying information. The processor may be configured to verify that the user access code matches the unique access code and the user identifying information matches the first patient identifying information. The processor may be configured to request, in response to the verifying, a new username and password via the client application. The processor may be configured to associate the new username and password with the new account, the online service, the first patient identifying information, and the second patient identifying information.

In an aspect, the disclosure provides a method of registering a user with an online service. The method may include receiving from an authorized enrollment provider an account creation request for a user including a first user identifying information and second user identifying information for the online service. The method may include receiving, via the authorized enrollment provider, an indication of consent from the user. The method may include receiving an order for the online service from the authorized enrollment provider. The method may include generating a unique access code for the user. The method may include transmitting a message to the user based on the second user identifying information, the message including the unique access code. The method may include receiving, from a client application via a secure link, a user access code and user identifying information. The method may include verifying that the user access code matches the unique access code and the user identifying information matches the first user identifying information. The method may include requesting, in response to the verifying, a new username and password via the client application. The method may include associating the new username and password with the new account, the online service, the first user identifying information, and the second user identifying information.

Additional advantages and novel features relating to implementations of the present disclosure will be set forth in part in the description that follows, and in part will become more apparent to those skilled in the art upon examination of the following or upon learning by practice thereof.

### DESCRIPTION OF THE FIGURES

In the drawings:

FIG. 1 is a diagram of an example computer system for providing paperless onboarding system, in accordance with an implementation of the present disclosure.

FIG. 2 is an example message diagram illustrating example messages within the paperless onboarding system, in accordance with an implementation of the present disclosure.

FIG. 3 is an example user interface for an enrollment provider system to create an account, in accordance with an implementation of the present disclosure.

FIG. 4 is an example user interface for an enrollment provider system to enroll a user, in accordance with an implementation of the present disclosure.

FIG. 5 is an example user interface for an enrollment provider system to obtain user consent, in accordance with an implementation of the present disclosure.

FIG. 6 is an example client interface for activating an account, in accordance with an implementation of the present disclosure.

FIG. 7 is an example client interface for entering an access code, in accordance with an implementation of the present disclosure.

FIG. 8 is an example client interface for verifying a user identity, in accordance with an implementation of the present disclosure.

FIG. 9 is an example client interface for verifying personal information, in accordance with an implementation of the present disclosure.

FIG. 10 is an example client interface for creating a user account, in accordance with an implementation of the present disclosure.

FIG. 11 is a flowchart of an example method of registering a user with an online service, in accordance with an implementation of the present disclosure.

FIG. 12 is a schematic block diagram of an example application server, in accordance with an implementation of the present disclosure.

### DETAILED DESCRIPTION

The present disclosure provides systems and methods for onboarding a user into an online service such as a digital therapeutic. A digital therapeutic may refer to a computer service that provides treatment for a medical condition. For example, a digital therapy may be intended to provide a patient access to therapy tools used during treatment sessions to improve recognized treatment outcomes. A digital therapeutic may also be referred to as a computerized behavioral therapy device. A computerized behavioral therapy device may be a prescription-only device intended to provide a computerized version of condition-specific behavioral therapy as an adjunct to clinician supervised outpatient treatment to patients with psychiatric conditions. A computerized behavioral therapy device for psychiatric disorders may be a Class II, prescription-only device. That is, a computerized behavioral therapy device may require a prescription or a medical order from a clinician. A wellness application may be a computer service that provides general health related functionality, but may not treat a specific medical condition.

Onboarding for a digital therapeutic may involve collection of patient information. In particular, because the digital therapeutic may be considered a prescription-only device, onboarding for the digital therapeutic may include transfer of patient information from a health care provider to a service provider and subsequent health insurance verification. Such transfer may be subject to restrictions on PHI and may require patient consent. Additionally, the onboarding process may be based on a prescription and satisfy requirements such as a National Council for Prescription Drug Programs (NCPDP) standard.

In an aspect, the present disclosure provides methods and systems for enrolling a user in an online service such as a digital therapeutic. The system may facilitate a method of enrollment involving communications between a provider device, an application server, and a client device. The provider device may collect information entered by a health care provider or stored in a health care provider system including first user information and second user information. The first user information may be patient identifying information such as name, date of birth, or sex. The second user information may be patient contact information such as mobile phone number, email address, caretaker, and emergency contact. The provider device may also obtain consent from the patient to participate in the digital therapeutic. The provider device may order the digital therapeutic, for example, as an electronic prescription or medical order.

The application server may receive the first and second information from the provider device and create an account for the user. The application server may generate a unique access code for the patient and send the unique access code to a client device using the second patient information. The client device may download and install a client application. The patient may enter the unique access code into the client application to activate the previously created account. The patient may enter the first user information into the client application to verify the patient. The patient may then use the client application to create a username and password for the account. The client application may then obtain the service (i.e., the digital therapeutic) from the application server and the patient may participate in the digital therapeutic via the client application.

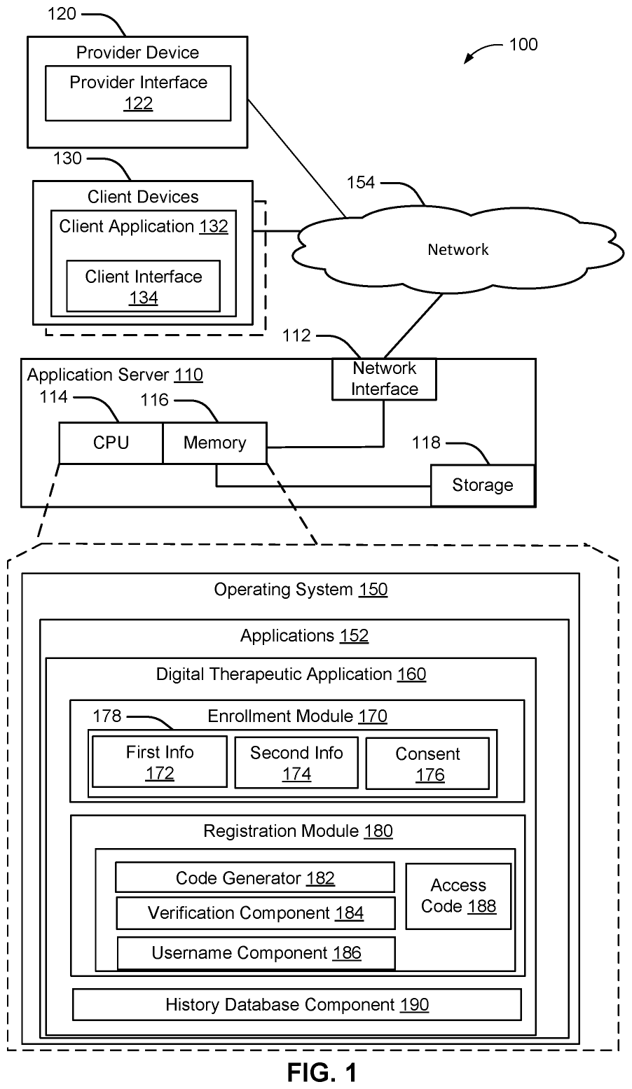

Referring now to FIG. 1, an example paperless onboarding system 100 includes a central application server 110 and a plurality of user devices including at least one provider device 120, and a plurality of client devices 130. The application server 110 may be, for example, any mobile or fixed computer device including but not limited to a computer server, desktop or laptop or tablet computer, a cellular telephone, a personal digital assistant (PDA), a handheld device, any other computer device having wired and/or wireless connection capability with one or more other devices, or any other type of computerized device capable of processing communications related data. In an aspect, the application server 110 may be implemented as one or more virtual machines hosted by a web services provider.

In an aspect, the paperless onboarding system 100 may include a digital therapeutic application 160 executed by the application server 110 that the paperless onboarding system 100 operates to onboard a user at one of the client devices 130 for providing an online service such as a digital therapeutic.

The digital therapeutic application 160 may include an enrollment module 170 configured to obtain information from an authorized enrollment provider (such as a health care provider) regarding a patient to enroll in the online service. In particular, the enrollment module 170 may obtain first information 172, second information 174, and consent 176. The first information 172 may be patient identifying information. For example, the first information 172 may include a name, a sex, a date of birth. In some implementations, the first information 172 may include a patient identifier, which may be, for example, a national identification number, a social security number, an insurance number, or other global unique identifier. The second information 174 may be patient contact information. For example, the second information 174 may include a mailing address, a phone number, or an email address. The consent 176 may be an indication of consent provided by the patient. For example, the consent 176 may include a digital signature or a digital document including a signature.

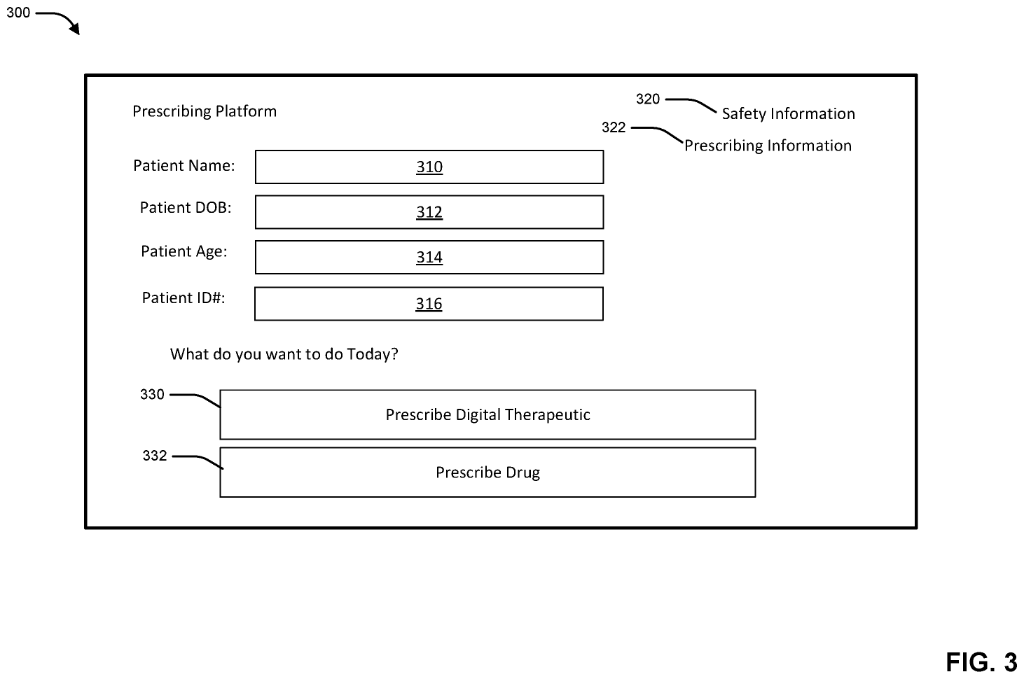

The enrollment module 170 may communicate with the provider interface 122. In an aspect, the enrollment module 170 may provide the user interfaces illustrated in FIGS. 3-5.

The digital therapeutic application 160 may include a registration module 180 that registers a client device 130 to participate in a digital therapeutic. The registration module 180 may include a code generator 182, a verification component 184, and a username component 186. The code generator 182 may generate an access code that allows a user that has been enrolled by the authorized enrollment provider to participate in the digital therapeutic. The access code may be generated for an account created by the enrollment module 170 and associated with the first info 172, the second info 174, and the consent 176. The access code may have a specified format. For instance, the access code may be exactly 8 digits, all numerals, with no more than two repeating numerals. The code generator 182 may provide the access code to the patient based on the second info 174. For instance, the code generator 182 may electrically send the access code to one or more contact numbers or addresses in the second info 174. For instance, the code generator 182 may send a text message (e.g., short message service (SMS) or multimedia message service (MMS) to a phone number included in the second info 174. As another example, the code generator 182 may place an automated call to the phone number and deliver the access code via a text to speech function. As yet another example, the code generator 182 may send an email with the access code to an email address in the second info 174.

A message including the access code may further provide instructions for obtaining the client application 132. For example, the message may provide a name of the client application 132 and identify one or more locations from which the client application 132 may be downloaded and installed. The client application 132, once downloaded and installed, may limit access to an authorization verification interface as illustrated in FIG. 7 and described in further detail below.

The verification component 184 may verify whether user identification information including a user access code matches information for a user account created by the authorized enrollment provider. For example, the verification component 184 may receive the user access code via the client application 132. The verification component 184 may also receive one or more pieces of user information that may correspond to first info 172. The verification component 184 may determine whether the user access code matches an access code generated by the code generator 182. The access codes may be unique, so a match may indicate that the user of the client device 130 received an access code. To ensure that the user is the patient authorized to participate in the digital therapeutic, the verification component 184 may compare the user information with the first info 172 stored in association with the access code. Accordingly, the verification component 184 may verify that the user is authorized to participate in the digital therapeutic.

The username component 186 may register a username and password with the previously created account. In an implementation, the usemame component 186 may receive a user generated username and password via the client application 132. In other implementations, the usemame component 186 may generate either the usemame or the password, which may be temporary.

The digital therapeutic application 160 may include a history database component 190. The history database component 190 may securely store information regarding treatment of the patient. In some implementations, the history database component 190 may generate sets of data for research. For example, the history database component 190 may determine whether a patient has consented to participation in research. The history database component 190 may anonymize stored data for patients that have consented to research.

The application server 110 may include a central processing unit (CPU) 114 that executes instructions stored in memory 116. For example, the CPU 114 may execute an operating system 150 and one or more applications 152, which may include digital therapeutic application 160. The application server 110 may also include a network interface 112 for communication with external devices via a network 154. For example, the application server 110 may communicate with a plurality of user devices including the provider device 120 and client devices 130.

Memory 116 may be configured for storing data and/or computer-executable instructions defining and/or associated with an operating system 150 and/or application 152, and CPU 114 may execute operating system 150 and/or applications 152. Memory 116 may represent one or more hardware memory devices accessible to application server 110. An example of memory 116 can include, but is not limited to, a type of memory usable by a computer, such as random access memory (RAM), read only memory (ROM), tapes, magnetic discs, optical discs, volatile memory, non-volatile memory, and any combination thereof. Memory 116 may store local versions of applications being executed by CPU 114. In an aspect, the memory 116 may include or communicate with a storage device 118, which may be a non-volatile memory.

The CPU 114 may include one or more processors for executing instructions. An example of CPU 114 can include, but is not limited to, any processor specially programmed as described herein, including a controller, microcontroller, application specific integrated circuit (ASIC), field programmable gate array (FPGA), system on chip (SoC), or other programmable logic or state machine. The CPU 114 may include other processing components such as an arithmetic logic unit (ALU), registers, and a control unit. The CPU 114 may include multiple cores and may be able to process different sets of instructions and/or data concurrently using the multiple cores to execute multiple threads. In an aspect, a graphics processing unit (GPU) may perform some operations of the CPU 114.

The operating system 150 may include instructions (such as applications 152) stored in memory 116 and executable by the CPU 114. The applications 152 may include a digital therapeutic application 160 configured to communicate with user devices via a respective interface (e.g., provider interface 122 or client interface 134). The digital therapeutic application 160 may provide the provider interface 122 that may be in communication with or otherwise operate in conjunction with a provider device 120. The provider interface 122 may be a graphical user interface (GUI) with which an end user may interact. For example, the provider interface 122 may be a web-page that is accessed through a browser application executed on the provider device 120. By loading the web-page, the browser application may effectively operate as a user interface for an application executed on the application server 110 (e.g., in the case of a web server). As another example, the provider interface 122 may be an application or operating system that runs on the provider device 120.

The digital therapeutic application 160 may also provide the client interface 134 that may be in communication with or otherwise operate in conjunction with a client device 130. The client interface 134 may be any user interface with which an end user may interact. For example, the client interface 134 may be a web-page that is accessed through a browser application (client) executed on the client device 130. By loading the web-page, the browser application may effectively operate as a user interface for an application executed on the application server 110 (e.g., in the case of a web server). Such an aspect may allow various types of user devices to serve as a client device 130 and participate in a digital therapeutic. For example, a communication session may include different types of client devices 130 such as desktop computers, laptop computers, tablets, and smart phones. In an aspect, the client interface 134 may be provided by a client application 132, which may be a stand-alone application installed on the client device 130.

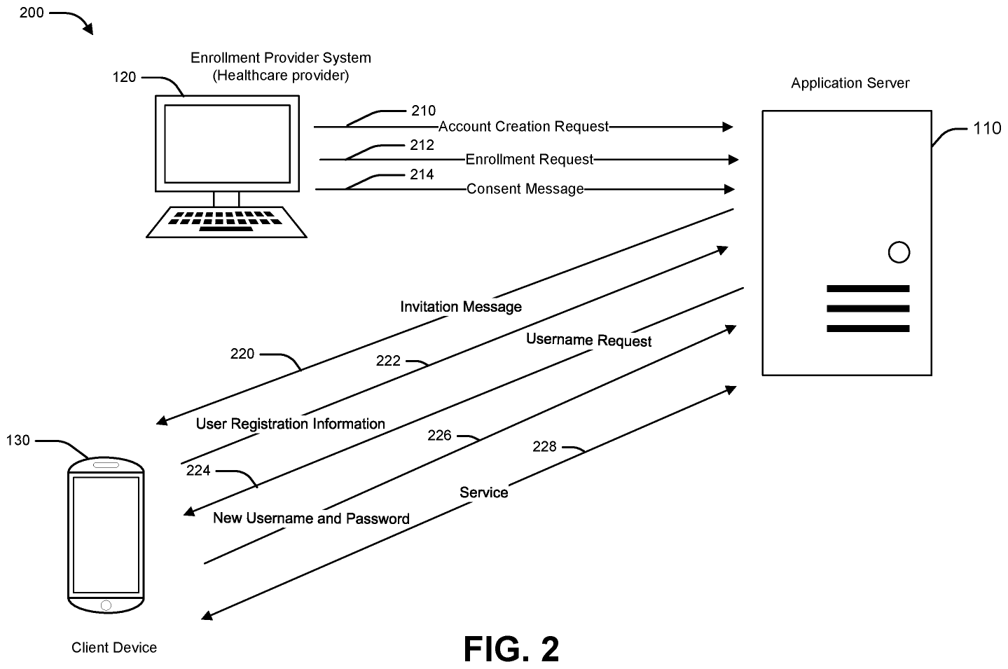

FIG. 2 is an example message diagram 200 illustrating example messages for onboarding a user in the paperless onboarding system 100. The enrollment provider device 120 may initiate the onboarding by transmitting an account creation request 210 to the application server 110. The account creation request 210 may include the first info 172. The application server 110 may generate an account 178 and store the first info 172 in association with the account. The application server 110 may also store an identification of the provider device 120 in association with the account 178. In an aspect, the account creation request 210 may not include PHI. The enrollment provider device 120 may transmit an enrollment request 212 to the application server 110. The enrollment request 212 may identify a specific digital therapeutic and include the second info 174. In some implementations, the enrollment request 212 may be an electronic prescription for a digital therapeutic. The enrollment request 212 may follow a prescription standard such as a NCPDP standard. For instance, the enrollment request 212 may include a NDC-like code for the digital therapeutic. The enrollment request 212 may be considered PHI because the enrollment request 212 links personal identifying information to a treatment for a condition. In an aspect, the account creation request 210 and the enrollment request 212 may be combined into a single request that may be considered PHI. The provider device 120 may transmit a consent message 214. The consent message 214 may include the consent 176.

In response to receiving the account creation request 210, the enrollment request 212, and the consent message 214, the application server 110 may transmit the invitation message 220 to the client device 130 based on the second info 174. In an aspect, the invitation message 220 may be a text message such as an SMS message or a MMS message that is sent to a phone number in the second info 174. The invitation message 220 may include the access code 188 generated by the code generator 182. The invitation message 220 may provide instructions for obtaining the client application 132.

The client device 130 may transmit the user registration information 222 via the client application 132. The client application 132 executing on the client device 130 may provide the client interface 134 to a user of the client device 130. The client interface 134 may prompt the user to enter a user access code. The user registration information 222 may include the user access code. The application server 110 (e.g., the verification component 184) may compare the user access code to the access code 188 to verify that the user was invited to receive the digital therapeutic. Similarly, the client interface 134 may prompt the user of the client device 130 to enter user identifying information. For example, the user identifying information may include one or more fields corresponding to the first info 172 such as a date of birth, name, or identification number. The application server 110 (e.g., the verification component 184) may compare the user identifying information to the first info 172 to verify that the user is the patient for whom the account 178 was created.

In response to verifying the user, the application server 110 may transmit a username request 224. The username request 224 may cause the client application 132 to prompt the user for a new username and password. The client application 132 may transmit the new username and password message 226 to the application server 110. The application server 110 may associate the new username and password with the account 178. The username and password may then be used to provide the service 228 between the client device 130 and the application server 110.

FIG. 3 is an example user interface 300 for an enrollment provider system to create an account. The user interface 300 may be a user interface of a prescribing platform. The prescribing platform may be provided by the application server 110 and the digital therapeutic application 160 (e.g., the enrollment module 170). The user interface 300 may include fields for the first info 174. For example, the user interface 300 may include a patient name field 310, a patient date of birth (DOB) field 312, a patient age field 314, and a patient ID number field 316. The fields may allow an operator (e.g., a clinician) to enter the requested information into a text field or select a value from a menu. In some implementations, the fields may be automatically populated with information from a patient management system executing on the provider device 120.

The user interface 300 may include links to information regarding the prescribing platform or a product. For example, the user interface 300 may include a link 320 to safety information and a link 322 to prescribing information.

The user interface 300 may provide one or more selectable buttons to select a product. For example, the user interface 300 may include a button 330 to prescribe a digital therapeutic. The user interface 300 may include a button 330 for each digital therapeutic available through the prescribing platform. In an aspect, the user interface 300 may include one or more buttons 332 to prescribe a drug. When the operator (e.g., clinician) selects the button 330, the user interface 300 may generate the account creation request 210 including the first info 172 entered in the patient information fields 310, 312, 314, 316.

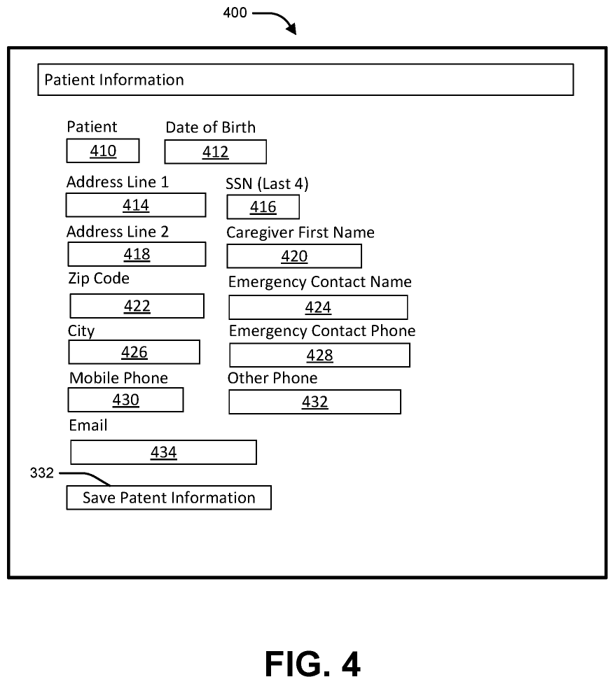

FIG. 4 is an example user interface 400 for an enrollment provider system to enroll a user. The user interface 400 may allow the operator (e.g., clinician) at the provider device 120 to enter patient information into one or more fields. The user interface 400 may include a patient field 410, a date of birth field 412, a first address field 414, a social security number field 416, a second address field 418, a caregiver first name field 420, a zip code field 422, an emergency contact name field 424, a city field 426, an emergency contact phone number field 428, a mobile phone field 430, an other phone field 432, an email field 434, and a save patient information button 332. One or more fields may be prefilled based on information collected from the user interface 300 (e.g., patient name field 410 and date of birth field 412).

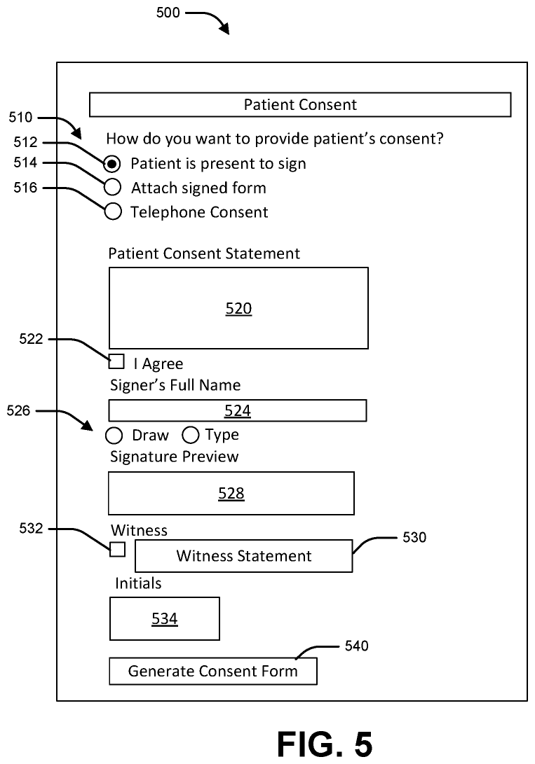

FIG. 5 is an example user interface 500 for an enrollment provider system to obtain user consent. The user interface 500 may include selectable options 510 for obtaining consent. For example, the options 510 may include a patient is present option 512, an attached signed form option 514, and a telephone consent option 516. The operator may select a radio button corresponding to the option. In some implementations, selection of one of the options 510 may change the appearance and other fields of the user interface 500. For example, the user interface 500 may illustrate the patient is present option 512 being selected. The user interface 500 may include a patient consent statement 520, which may include a statement specific to a selected digital therapeutic. The consent statement 520 may also include a statement of consent to receive communications via one or more means (e.g., mobile phone or email). The user interface 500 may include a checkbox 522 to be selected by a patient using the provider device 120 to acknowledge the consent statement 520. The user interface 500 may include a name field 524, which may include the full name of the patient based on user interface 300 and/or 400. The user interface 500 may include options 526 to input a signature (e.g., type or draw), which may appear in a signature box 528. The user interface 500 may include a witness statement 530 indicating that a witness observed the patient sign the consent statement and a checkbox 532 to acknowledge the witness statement 530. The user interface 500 may include an initials field 534 for the witness to initial the witness statement 530. The user interface 500 may include a generate consent form button 540 that generates a digital document based on the user interface 500 to record the patient's consent. The digital document may include a digital signature that allows authentication of the digital document.

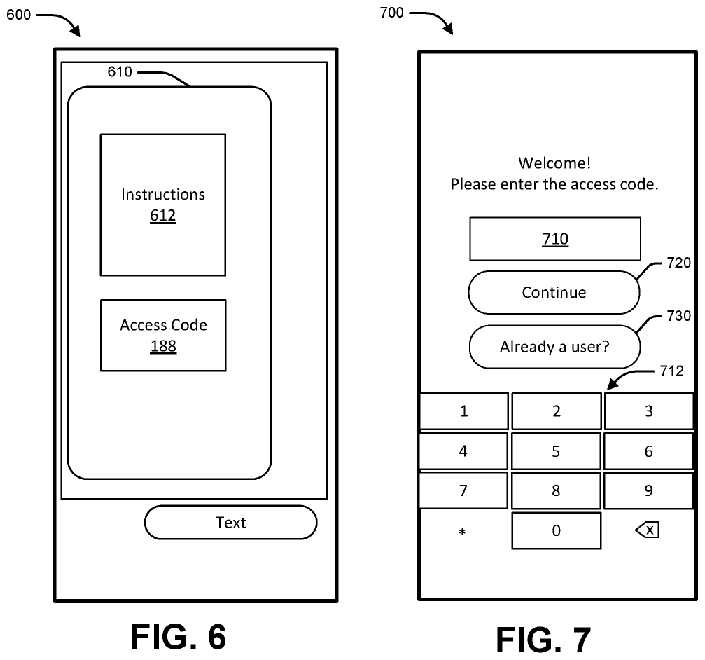

FIG. 6 is an example client interface 600 for activating an account. The client interface 600 may be a user interface for a mobile device. The client interface 600 may display a message 610. The message 610 may be, for example, a SMS, MMS, or email. The message 610 may include instructions 612 and an access code 188. The instructions 612 may provide instructions for obtaining the client application 132 from one or more application services. The instructions 612 may include a hyperlink to download the client application 132. The access code 188 may be the unique access code generated specifically for a patient in response to receipt of the first info 172. The message 610 may be sent to a number or address indicated in the second info 174. The first info 172 and the second info 174 may be associated with a patient account at the application server 110. Accordingly, the access code 188 may be used only by the enrolled user to access the patient account.

FIG. 7 is an example client interface 700 for entering an access code (e.g., access code 188). The client interface 700 may be provided by the client application 132. The client interface 700 may be the only interface accessible in the client application 132 prior to a user associating a local copy of the client application 132 with a registered account. That is, the client application 132 may block access to one or more functions until the local copy of the client application 132 is linked to the registered account. The client interface 700 may include a field 710 for displaying an entered access code. The client interface 700 may include a digital number pad 712 for a user to enter the access code. The client interface 700 may include a continue button 720 that causes the client application 132 to transmit the entered access code to the application server 110 (e.g., user registration information 222). In some implementations, the client interface 700 may include a login button 730 that may allow a user with an active account to enter a usemame and password instead of an access code.

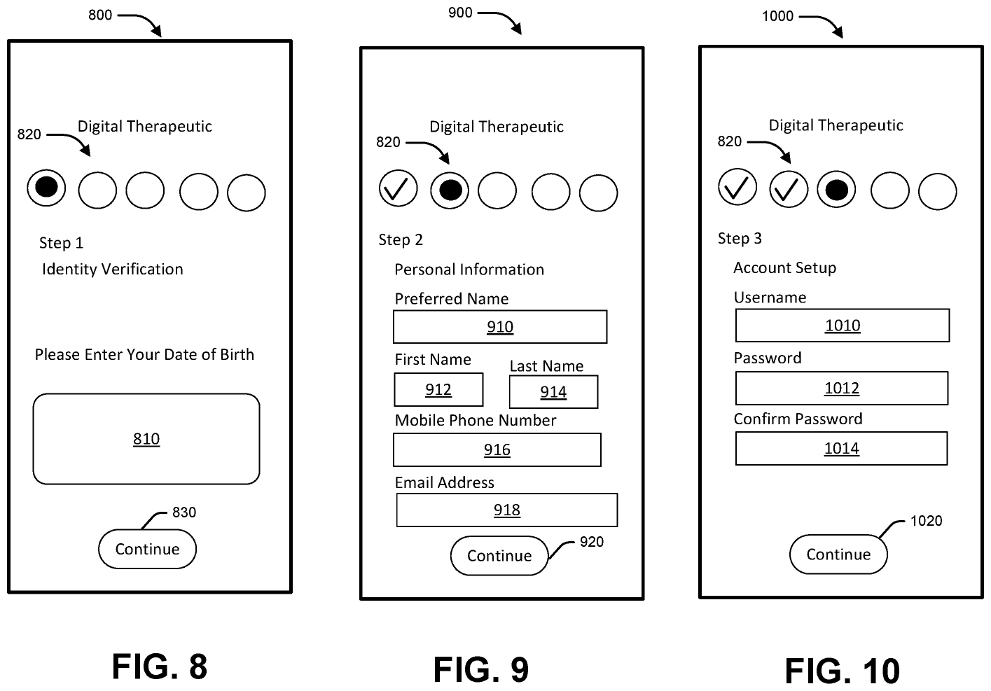

FIG. 8 is an example client interface 800 for verifying a user identity. The client interface 800 may be provided by the client application 132. The client interface 800 may include a field 810 for a user to enter one or more pieces of information corresponding to first info 172. For example, in an aspect, the date of birth may be used to verify that the user is the patient for whom an account was created. The field 810 may include a list of selectable dates. In aspects where additional pieces of information are used for verification, the client interface 800 may include corresponding fields. The client interface 800 may include a progress indicator 820 that indicates a current step of a registration process. The client interface 800 may include a continue button 830 that may cause the client application 132 to send the information in the field 810 to the application server 110 and present a next client interface (e.g., client interface 900).

FIG. 9 is an example client interface 900 for verifying personal information. The client interface 900 may be presented by the client application 132. The client interface 900 may include a preferred name field 910, a first name field 912, a last name field 914, a mobile phone number field 916, and an email address field 918. The fields of the client interface 900 may be pre-populated based on information from the user interfaces 300 and/or 400. For example, the preferred name field 910, first name field 912, and last name field 914 may each include a portion of the patient name field 310. The mobile phone number field 916 may correspond to the mobile phone number field 430, and the email address field 918 may correspond to the email field 434. The client interface 900 may allow the user to edit any of the fields 910, 912, 914, 916, or 918. The client interface 900 may include the progress indicator 820, which may be updated to show a second step of a registration process. The client interface 900 may include a continue button 920, which may cause the client application 132 to send the content of the fields 910, 912, 914, 916, or 918 to the application server 110 present a next client interface (e.g., client interface 1000).

FIG. 10 is an example client interface 1000 for creating a user account. The client interface 1000 may be presented by the client application 132. The client interface 1000 may include a usemame field 1010, a password field 1012, and a confirm password field 1014. The client interface 1000 may receive input from the user in each of the fields 1010, 1012, and 1014. The client interface 1000 may include the progress indicator 820, which may indicate a third step of a registration process. The client interface 1000 may include a continue button 1020, which may cause the client application 132 to send the information in the fields 1010, 1012, and 1014 to the application server 1010 (e.g., as new username and password message 226).

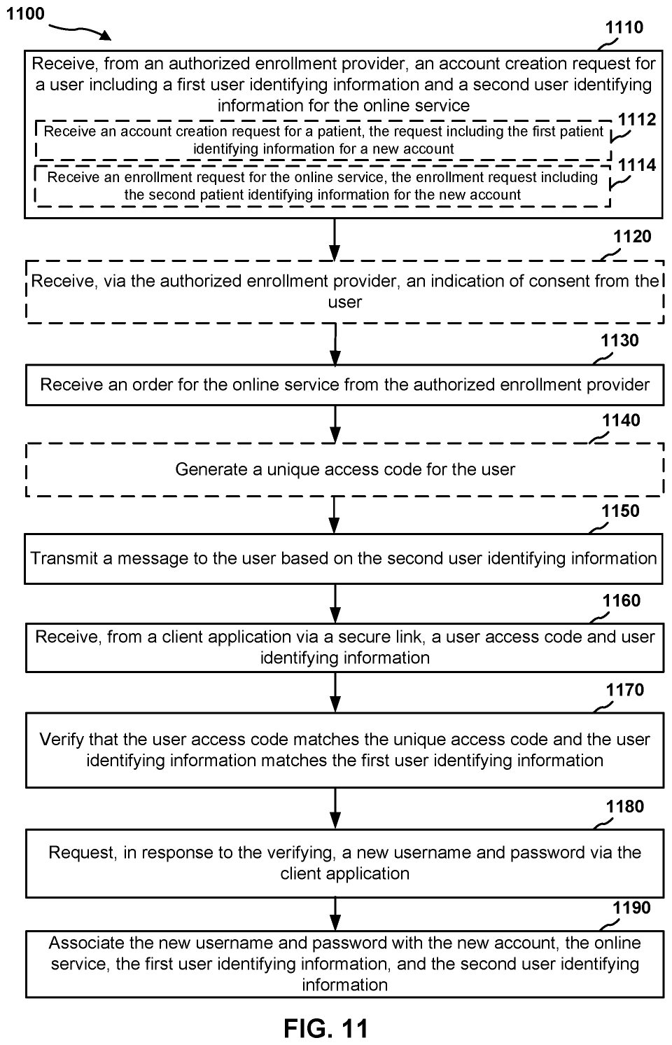

FIG. 11 is a flowchart of an example method 1100 of controlling access to a live video session. The method 1100 may be performed by the application server 110 including the CPU 114 executing the digital therapeutic application 160. The application server 110 may be in communication with a provider device 120 and a client device 130.

At block 1110, the method 1100 may include receiving from an authorized enrollment provider an account creation request for a user including a first user identifying information and second user identifying information for the online service. For example, the application server 110 (e.g., enrollment module 170) may receive, from the provider device 120, the account creation request 210 for a user. For instance, the user may be a patient and the authorized enrollment provider may be a healthcare provider such as a clinician. The account creation request 210 may include the first info 172 and/or the second info 174. For instance, the first info 172 may be first patient identifying information including one or more of: a name, a sex, or a date of birth. The second info 174 may be second patient identifying information and may include one or more of: a mailing address, a phone number, or an email address. The enrollment module 170 may generate a new user account 178 and store the first info 172 and the second info 174 in association with the new user account 178. In an aspect, the account creation request 210 may be send as a single message, while in another aspect, the first info 172 and the second info 174 may be sent separately. For instance, in sub-block 1112, the block 1110 may optionally include receiving an account creation request 210 for a patient, the request including the first patient identifying information for a new account. In sub-block 1114, the block 1110 may optionally include receiving an enrollment request 212 for the online service, the enrollment request including the second patient identifying information for the new account.

At block 1120, the method 1100 may optionally include receiving, via the authorized enrollment provider, an indication of consent from the user. For example, the application server 110 may receive consent message 214 from the provider device 120. The consent message 214 may be generated via the user interface 500.

At block 1130, the method 1100 may optionally include receiving an order for the online service from the authorized enrollment provider. For example, the application server 110 may receive a separate enrollment request 212. The enrollment request 212 may be an electronic prescription and the online service may be a prescription-only digital therapeutic. The enrollment request 212 may be generated by the user interface 300 (e.g., in response to selection of button 330). In some implementations, the enrollment request 212 may include a NDC-like code.

At block 1140, the method 1100 may include generating a unique access code for the user. For example, the application server 110 (e.g., code generator 182) may generate the access code 188. For instance, the code generator 182 may implement rules that ensure the access code 188 is unique and has certain properties (e.g., a fixed length and a limited number of repeating numerals).

At block 1150, the method 1100 may include transmitting a message to the user based on the second user identifying information, the message including the unique access code. For example, the application server 110 may transmit the invitation message 220. The invitation message 220 may include the access code 188. As illustrated in FIG. 6, the invitation message (e.g., message 610) may include instructions 612 for obtaining a client application 132. In an aspect, transmitting the invitation message 220 may be in response to receiving the indication of consent in block 1120. That is, the application server 110 may transmit the invitation message 220 when the consent message 214 is received and the consent 176 is associated with the account 178.

At block 1160, the method 1100 may include receiving, from a client application via a secure link, a user access code and user identifying information. For example, the application server 110 (e.g., verification component 184) may receive the user registration information 222, which may include a user access code entered into field 710 of the client interface 700 and the user identifying information (e.g., date of birth) entered into field 810 of the client interface 800.

At block 1170, the method 1100 may include verifying that the user access code matches the unique access code and the user identifying information matches the first user identifying information. For example, the application server 110 (e.g., verification component 184) may verify that the user access code matches the access code 188 and that the user identifying information (e.g., field 810) matches the first info 172 (e.g., field 312).

At block 1180, the method 1100 may include requesting, in response to the verifying, a new username and password via the client application. For example, the application server 110 (e.g., username component 186) may request, in response to block 1170, a new username and password via the client application 132. For example, the application server 110 may transmit the username request 224, which may cause the client application 132 to present the client interface 1000 (FIG. 10).

At block 1190, the method 1100 may include associating the new username and password with the new account, the online service, the first user identifying information, and the second user identifying information. For example, the application server 110 (e.g., username component 186) may receive the username and password message 226. The username component 186 may add the username and password to the account 178 created for the user by the enrollment module 170. In some implementations, in response to associating the new username and password with the new account, the application server 110 may retire the access code 188. That is, the access code 188 may be used for only one user account and not repeated.

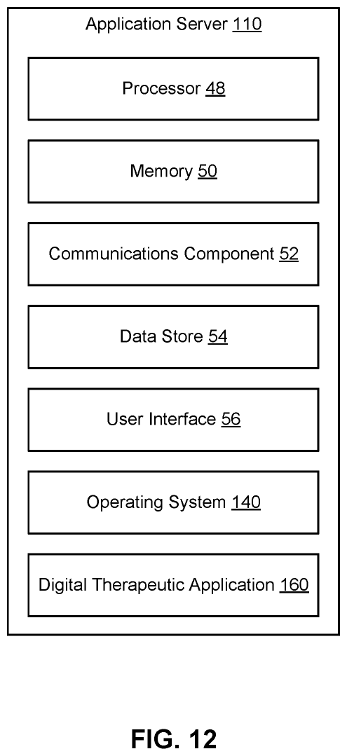

Referring now to FIG. 12, illustrated is an example application server 110 in accordance with an aspect, including additional component details as compared to FIG. 1. In one example, application server 110 may include processor 48 for carrying out processing functions associated with one or more of components and functions described herein. Processor 48 can include a single or multiple set of processors or multi-core processors. Moreover, processor 48 can be implemented as an integrated processing system and/or a distributed processing system. In an aspect, for example, processor 48 may include CPU 114.

In an example, application server 110 may include memory 50 for storing instructions executable by the processor 48 for carrying out the functions described herein. In an aspect, for example, memory 50 may include memory 116. The memory 50 may include instructions for executing the digital therapeutic application 160.

Further, application server 110 may include a communications component 52 that provides for establishing and maintaining communications with one or more parties utilizing hardware, software, and services as described herein. Communications component 52 may carry communications between components on c application server 110, as well as between application server 110 and external devices, such as devices located across a communications network 154 and/or devices serially or locally connected to application server 110. For example, communications component 52 may include one or more buses, and may further include transmit chain components and receive chain components associated with a transmitter and receiver, respectively, operable for interfacing with external devices.

Additionally, application server 110 may include a data store 54, which can be any suitable combination of hardware and/or software, that provides for mass storage of information, databases, and programs employed in connection with aspects described herein. For example, data store 54 may be a data repository for operating system 150 and/or applications 152. The data store may include memory 116 and/or storage device 118.

Application server 110 may also include a user interface component 56 operable to receive inputs from a user of application server 110 and further operable to generate outputs for presentation to the user. User interface component 56 may include one or more input devices, including but not limited to a keyboard, a number pad, a mouse, a touch-sensitive display, a digitizer, a navigation key, a function key, a microphone, a voice recognition component, any other mechanism capable of receiving an input from a user, or any combination thereof. Further, user interface component 56 may include one or more output devices, including but not limited to a display, a speaker, a haptic feedback mechanism, a printer, any other mechanism capable of presenting an output to a user, or any combination thereof

In an aspect, user interface component 56 may transmit and/or receive messages corresponding to the operation of operating system 150 and/or applications 152. In addition, processor 48 may execute operating system 150 and/or applications 152, and memory 50 or data store 54 may store them.

As used in this application, the terms “component,” “system” and the like are intended to include a computer-related entity, such as but not limited to hardware, firmware, a combination of hardware and software, software, or software in execution. For example, a component may be, but is not limited to being, a process running on a processor, a processor, an object, an executable, a thread of execution, a program, and/or a computer. By way of illustration, both an application running on a computer device and the computer device can be a component. One or more components can reside within a process and/or thread of execution and a component may be localized on one computer and/or distributed between two or more computers. In addition, these components can execute from various computer readable media having various data structures stored thereon. The components may communicate by way of local and/or remote processes such as in accordance with a signal having one or more data packets, such as data from one component interacting with another component in a local system, distributed system, and/or across a network such as the Internet with other systems by way of the signal.

Moreover, the term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

Various aspects or features may have been presented in terms of systems that may include a number of devices, components, modules, and the like. A person skilled in the art should understand and appreciate that the various systems may include additional devices, components, modules, etc. and/or may not include all of the devices, components, modules etc. discussed in connection with the figures. A combination of these approaches may also be used.

The various illustrative logics, logical blocks, and actions of methods described in connection with the embodiments disclosed herein may be implemented or performed with a specially-programmed one of a general purpose processor, a GPU, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computer devices, e.g., a combination of a DSP and a microprocessor, a plurality of microprocessors, one or more microprocessors in conjunction with a DSP core, or any other such configuration. Additionally, at least one processor may comprise one or more components operable to perform one or more of the steps and/or actions described above.

Further, the steps and/or actions of a method or procedure described in connection with the aspects disclosed herein may be embodied directly in hardware, in a software module executed by a processor, or in a combination of the two. A software module may reside in RAM memory, flash memory, ROM memory, EPROM memory, EEPROM memory, registers, a hard disk, a removable disk, a CD-ROM, or any other form of storage medium known in the art. An exemplary storage medium may be coupled to the processor, such that the processor can read information from, and write information to, the storage medium. In the alternative, the storage medium may be integral to the processor. Further, in some aspects, the processor and the storage medium may reside in an ASIC. Additionally, the ASIC may reside in a user terminal. In the alternative, the processor and the storage medium may reside as discrete components in a user terminal. Additionally, in some aspects, the steps and/or actions of a method or procedure may reside as one or any combination or set of codes and/or instructions on a machine readable medium and/or computer readable medium, which may be incorporated into a computer program product.

In one or more aspects, the functions described may be implemented in hardware, software, firmware, or any combination thereof. If implemented in software, the functions may be stored or transmitted as one or more instructions or code on a computer-readable medium. Computer-readable media includes both computer storage media and communication media including any medium that facilitates transfer of a computer program from one place to another. A storage medium may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, includes compact disc (CD), laser disc, optical disc, digital versatile disc (DVD), floppy disk and Blu-ray disc where disks usually reproduce data magnetically, while discs usually reproduce data optically with lasers. Combinations of the above should also be included within the scope of computer-readable media.

While aspects of the present disclosure have been described in connection with examples thereof, it will be understood by those skilled in the art that variations and modifications of the aspects described above may be made without departing from the scope hereof. Other aspects will be apparent to those skilled in the art from a consideration of the specification or from a practice in accordance with examples disclosed herein.

What is claimed is:

1. A method of registering a user with an online service, comprising:
receiving, from a health care provider, first patient identifying information for a new account and second patient identifying information for the new account;
transmitting a message to the patient based on the second patient identifying information, the message including a unique access code for the patient;
receiving, from a client application via a secure link, a user access code and user identifying information;
verifying that the user access code matches the unique access code and the user identifying information matches the first patient identifying information;
requesting, in response to the verifying, a new username and password via the client application; and
associating the new username and password with the new account, the online service, the first patient identifying information, and the second patient identifying information.

2. The method of claim 1, wherein the first patient identifying information includes one or more of: a name, a sex, or a date of birth.

3. The method of claim 1, wherein the second patient identifying information includes one or more of: a mailing address, a phone number, or an email address.

4. The method of claim 1, further comprising receiving, via the health care provider, an indication of consent from the patient, wherein transmitting the message is in response to the consent from the patient.

5. The method of claim 4, wherein the indication of consent is one of a signed digital form, a digital copy of a signed form, or a recording of a telephone consent.

6. The method of claim 1, wherein the unique access code is eight digits with no more than two repeating numbers.

7. The method of claim 1, further comprising retiring the unique access code in response to confirming acceptance of the new username and password.

8. The method of claim 1, wherein receiving the patient information comprises:
receiving from a health care provider an account creation request for a patient, the request including the first patient identifying information for a new account; and
receiving, from a health care provider, an enrollment request for the online service, the enrollment request including the second patient identifying information for the new account.

9. The method of claim 8, wherein the enrollment request is an electronic prescription and the online service is a prescription-only digital therapeutic.

10. An apparatus for providing an online service to registered users, comprising:
a processor; and
a memory storing computer-executable instructions that when executed by the processor, cause the processor to:
receive, from a health care provider, first patient identifying information for a new account and second patient identifying information for the new account;
transmit a message to the patient based on the second patient identifying information, the message including a unique access code for the patient;
receive, from a client application via a secure link, a user access code and user identifying information;
verify that the user access code matches the unique access code and the user identifying information matches the first patient identifying information;
request, in response to the verifying, a new username and password via the client application; and
associate the new username and password with the new account, the online service, the first patient identifying information, and the second patient identifying information.

11. The apparatus of claim 10, wherein the first patient identifying information includes one or more of: a name, a sex, or a date of birth.

12. The apparatus of claim 10, wherein the second patient identifying information includes one or more of: a mailing address, a phone number, or an email address.

13. The apparatus of claim 10, wherein the processor is configured to receive, via the health care provider, an indication of consent from the patient, wherein the processor is configured to transmit the message is in response to the consent from the patient.

14. The apparatus of claim 13, wherein the indication of consent is one of a signed digital form, a digital copy of a signed form, or a recording of a telephone consent.

15. The apparatus of claim 10, wherein the unique access code is eight digits with no more than two repeating numbers.

16. The apparatus of claim 10, wherein the processor is configured to retire the unique access code in response to confirming acceptance of the new username and password.

17. The apparatus of claim 10, wherein the processor is configured to receive the patient information by:
receiving from a health care provider an account creation request for a patient, the request including the first patient identifying information for a new account; and
receiving, from a health care provider, an enrollment request for the online service, the enrollment request including the second patient identifying information for the new account.

18. The apparatus of claim 17, wherein the enrollment request is an electronic prescription and the online service is a prescription-only digital therapeutic.

19. A method of registering a user with an online service, comprising:
receiving from an authorized enrollment provider an account creation request for a user including a first user identifying information and second user identifying information for the online service;
receiving, via the authorized enrollment provider, an indication of consent from the user;
receiving an order for the online service from the authorized enrollment provider;
generating a unique access code for the user;
transmitting a message to the user based on the second user identifying information, the message including the unique access code;
receiving, from a client application via a secure link, a user access code and user identifying information;
verifying that the user access code matches the unique access code and the user identifying information matches the first user identifying information;
requesting, in response to the verifying, a new username and password via the client application; and
associating the new username and password with the new account, the online service, the first user identifying information, and the second user identifying information.
